(19) United States
(12) Patent Application Publication
(10) Pub. No.: US 2004/0225272 A1
(43) Pub. Date: Nov. 11, 2004

(54) Tampon with joined skirt portion

(75) Inventors: Nancy Karapasha, Cincinnati, OH (US); Dean Jeffrey Daniels, Cincinnati, OH (US)

Correspondence Address: THE PROCTER & GAMBLE COMPANY, INTELLECTUAL PROPERTY DIVISION, WINTON HILL TECHNICAL CENTER - BOX 161, 6110 CENTER HILL AVENUE, CINCINNATI, OH, 45224, US

(73) Assignee: The Procter & Gamble Company

(21) Appl. No.: 10/430,916

(22) Filed: May 7, 2003

(51) Int. Cl. A61F 13/15; A61F 13/20

(52) U.S. Cl. 604/385.17

(57) ABSTRACT

The present invention relates to a catamenial tampon with a joined skirt portion. The tampon includes an absorbent material having a first surface opposed to the second surface and an insertion end opposed to a withdrawal end. The overwrap covers at least a portion of the first surface and the second surface proximal to the withdrawal end of the absorbent material. The overwrap extends beyond the withdrawal end of the first surface of the absorbent material to define a first skirt portion. The overwrap also extends beyond the withdrawal end of the second surface of the absorbent material to define a second skirt portion. The first skirt portion is joined to the second skirt portion proximal to the withdrawal end of the absorbent.

### FIELD OF THE INVENTION

The present invention relates to an improved absorbent catamenial tampon having increased leakage protection.

### BACKGROUND OF THE INVENTION

A wide variety of absorbent catamenial tampons have long been known in the art. While it has been found that these tampons perform their intended function tolerably well, even the best of them do not always re-expand sufficiently, or fast enough, to provide good coverage against leakage. One common problem with tampons is “bypass” failure that occurs when the menses travels along the length of the vagina without contacting the tampon, i.e., the tampon fails to intercept the flowing menses. During a tampon change, some residual menses may be left near the introitus of the vagina. This may be fluid which was previously absorbed, but which subsequently “squeezed out” of the tampon as it was withdrawn through the sphincter of the vagina. Such residual fluid, particularly if located near the introitus (i.e., in the lower vaginal cavity) may not be effectively absorbed by the replacement tampon. Secondary absorbents and skirts have been used to absorb bypassed fluid from the lower vaginal vault. However, some of these mechanisms have problems with maintaining integrity and effectiveness during use. It is believed that the superior design of the present invention comprising the use of a skirt portion that is joined together will both improve the fluid handing properties of the tampon as well as, improve the integrity of the tampon as a whole during use.

### BACKGROUND ART

U.S. Pat. No. 6,186,995 issued to John M. Tharpe on Feb. 13, 2001 relates to VAGINAL TAMPON AND METHOD FOR FABRICATION THEREOF.

### SUMMARY OF THE INVENTION

The present invention relates to a catamenial tampon with a joined skirt portion. The tampon comprises an absorbent material having a first surface opposed to a second surface and an insertion end opposed to a withdrawal end. The overwrap covers at least a portion of the first surface and at least a portion of the second surface proximal to the withdrawal end of the absorbent material. The overwrap extends beyond the withdrawal end of the first surface of the absorbent material to define a first skirt portion. The overwrap also extends beyond the withdrawal end of the second surface of said absorbent material to define a second skirt portion. The first skirt portion is joined to the second skirt portion proximal to the withdrawal end of the absorbent material.

### BRIEF DESCRIPTION OF THE DRAWINGS

While the specification concludes with claims particularly pointing out and distinctly claiming the subject matter that is regarded as forming the present invention, it is believed that the invention will be better understood from the following description taken in conjunction with the accompanying drawings, in which:

FIG. 1 is a plan view of the absorbent material and overwrap prior to compression.

FIG. 2 is a longitudinal cross section of the absorbent material and overwrap prior to compression.

FIG. 3 is a longitudinal cross section of the absorbent material and two overwraps prior to compression.

FIG. 4 is a plan view of the absorbent material with the overwrap wrapped in the direction of the transverse axis around the absorbent material.

FIG. 5 is a plan view of the absorbent material with two overwraps wrapped in the direction of the transverse axis around the absorbent material.

### DETAILED DESCRIPTION OF THE INVENTION

As used herein “compression” refers to the process of pressing, squeezing, compacting or otherwise manipulating the size, shape, and/or volume of a material to obtain a tampon having a vaginally insertable shape. The term “compressed” refers to the state of a material or materials subsequent to compression. Conversely, the term “uncompressed” refers to the state of a material or materials prior to compression. In some embodiments, uncompressed tampons can be utilized in vivo. The term “compressible” is the ability of a material to undergo compression.

The term “folded” as used herein, is the configuration of the compressed absorbent member that may be incidental to lateral compression of the absorbent material or may purposely occur prior to a compression step. Such a configuration is readily recognizable, for example, when the absorbent material abruptly changes direction such that one part of the absorbent material bends and lies over another part of the absorbent material. When overwrap is placed on the absorbent material prior to compression, it too may be “folded.”

As used herein “hydrophilic” and “hydrophobic” have meanings well established in the art with respect to the contact angle of a drop of water on the surface of a material. For example, a material having a contact angle of greater than about 75 degrees may be considered hydrophobic, and a material having a contact angle of less than about 75 degrees may be considered hydrophilic. Absolute values of hydrophobicity/hydrophilicity are not generally important, but relative values are. Thus, in some embodiments, one overwrap or region of the overwrap may be more hydrophilic or hydrophobic than another overwrap or region of the overwrap.

The term “joined” or “attached” as used herein, encompasses configurations in which a first element is directly secured to a second element by affixing the first element directly to the second element; configurations in which the first element is indirectly secured to the second element by affixing the first element to intermediate member(s) which in turn are affixed to the second element; and configurations in which the first element is integral with the second element; i.e., the first element is essentially part of the second element.

As used herein, the term “non-absorbent” refers to a structure that does not retain a significant portion of fluid in its structure.

The term “rolled” as used herein, is the configuration of the compressed absorbent member after winding the absorbent material and the overwrap or overwraps in a spiral round and round upon itself As used herein the term “skirt” or “skirt portion” refers to the portion of the overwrap that extends beyond the withdrawal end of the absorbent material or compressed absorbent.

As used herein, “self-sustaining” is a measure of the degree or sufficiency to which the tampon retains its compressed form after stabilization such that in the subsequent absence of the external forces, the resulting tampon will tend to retain its vaginally insertable shape and size. For tampons, it is found that control of the level of moisture within the tampon is a factor for helping the tampon to retain its shape subsequent the absence of the external compression forces. It will be understood by one of skill in the art that this self-sustaining form need not, and preferably does not persist during actual use of the tampon. That is, once the tampon is inserted into the vagina or other body cavity and begins to acquire fluid, the tampon will begin to expand and may lose its self-sustaining form.

As used herein the term “tampon” refers to any type of absorbent structure that is inserted into the vaginal canal or other body cavities for the absorption of fluid therefrom, to aid in wound healing, or for the delivery of active materials, such as medicaments, or moisture. Tampons have a length, a width, a longitudinal axis and a radial axis. The tampon's length can be measured from the insertion end to the withdrawal end along the longitudinal axis. A typical compressed tampon is 30-60 mm in length. A tampon may be straight or non-linear in shape, such as curved along the longitudinal axis. The width of a tampon, unless otherwise stated in the specification, corresponds to the largest cylindrical cross-section along the length. A typical compressed tampon is 8-20 mm wide. The tampon may be compressed into a generally cylindrical configuration in the radial direction, axially along the longitudinal and lateral axes or in both the radial and axial directions. While the tampon may be compressed into a substantially cylindrical configuration, other shapes are possible. These may include shapes having a cross section that may be described as rectangular, triangular, trapezoidal, semi-circular, hourglass, or other suitable shapes.

As used herein the terms “vaginal cavity,” “within the vagina” and “vaginal interior,” are intended to be synonymous and refer to the internal genitalia of the human female in the pudendal region of the body and does not include the interlabial space, the floor of vestibule or the externally visible genitalia.

As used herein, “mm” is millimeters, and “g/m²” is grams per meter squared.

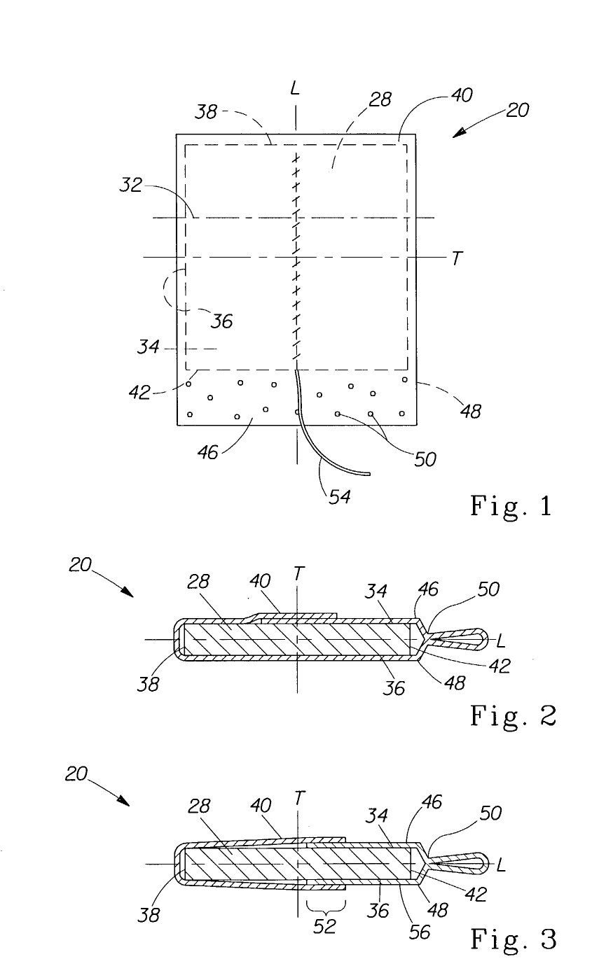

FIG. 1 shows a plan view of a tampon 20 of the present invention comprising the absorbent material 28 and an overwrap 40 prior to compression. The absorbent material 28 has a first surface 34 opposed to a second surface 36 and an insertion end 38 opposed to a withdrawal end 42. The absorbent material has both a longitudinal axis “L” and a transverse axis “T.” As shown in FIG. 1, an overwrap 40 substantially covers the first surface 34 and opposed second surface 36 of the absorbent material 28. The overwrap is wrapped around the absorbent material 28 so that the overwrap 40 is proximate with the insertion end 38 and extends beyond the withdrawal end 42. The overwrap 40 extends beyond the withdrawal end 42 of the first surface 34 of the absorbent material to form a first skirt portion 46. The overwrap 40 also extends beyond the withdrawal end 42 of the second surface 36 of the absorbent material to form a second skirt portion 48. The overwrap overlaps at the seam 32. In the embodiment shown, the first skirt portion 46 is joined to the second skirt portion 48 proximal to the withdrawal end 42 of the absorbent material 28 by spot bonds 50. The tampon 20 depicted in FIG. 1 includes a withdrawal means 54.

While several methods of making the tampon 20 of the present invention should be apparent to one of skill in the art in light of the disclosure herein, following is a description of some methods of making a tampon 20 of the present invention.

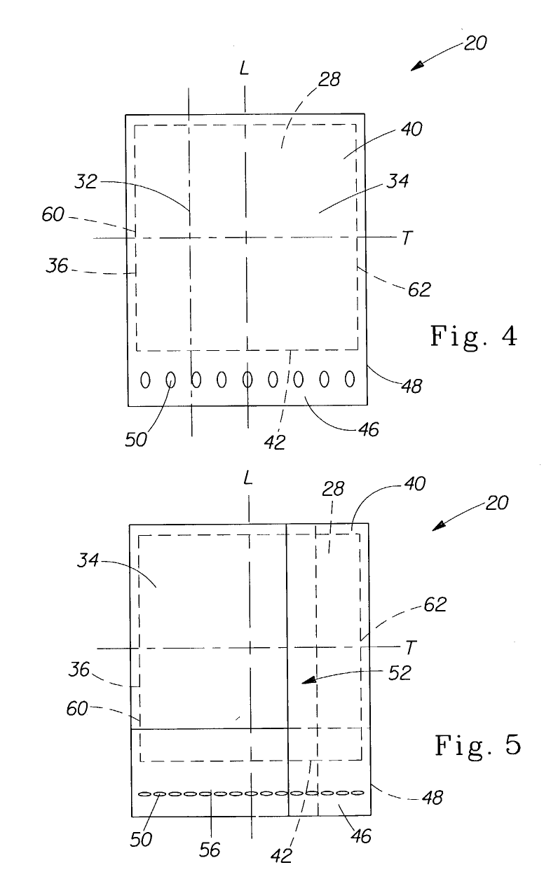

The overwrap 40 material may be wrapped in various configurations in the direction of longitudinal axis “L” as shown below in FIG. 2 and FIG. 3 or wrapped in the direction of the transverse axis “T,” as shown below in FIG. 4 and FIG. 5. Because overwraps 40 and 56 can be wrapped in the various configurations, the width and length of the overwrap 40, and 56 may vary. The width of the overwraps 40, and 56 may be wider or less wide than the measure of the longitudinal or transverse axis of the absorbent material it is being wrapped around. As well, the length of the overwraps 40, and 56 may be longer or shorter than the measure of the longitudinal axis “L” or transverse axis “T” of the absorbent material 28 it is being wrapped around.

In some embodiments of making the tampon 20 of the present invention, absorbent material 28 and one overwrap 40 is provided and wrapped around the absorbent material 28 in the direction of the longitudinal axis “L.” FIG. 2 shows a longitudinal cross section of the absorbent material 28 and overwrap 40 prior to compression. The absorbent material has a first surface 34 opposed to a second surface 36 and an insertion end 38 opposed to a withdrawal end 42. The absorbent material 28 is located in the center of the longitudinal cross-section and the overwrap 40 is wrapped in the direction of the longitudinal axis “L” extending on to and covering the first surface 34 and the opposed second surface 36 of the absorbent material 28. The overwrap 40 extends beyond the withdrawal end 42 of the first surface 34 of the absorbent material to form a first skirt portion 46. The overwrap 40 extends beyond the withdrawal end 42 of the second surface 36 of the absorbent material to form a second skirt portion 48. In the embodiment shown, the first skirt portion 46 is joined to the second skirt portion 48 proximal to the withdrawal end 42 of the absorbent material 28 by spot bonds 50.

In some embodiments of making the tampon 20 of the present invention, absorbent material 28 and two overwraps 40, 56 are provided and wrapped around the absorbent material 28 in the direction of the longitudinal axis “L.” FIG. 3 shows a longitudinal cross section of a tampon 20 of the present invention comprising absorbent material 28, and two overwraps 40 and 56 prior to compression. The absorbent material 28 has a first surface 34 opposed to the second surface 36. The absorbent material 28 is located in the center of the longitudinal cross-section between the overwrap 40 and the overwrap 56. One overwrap 40 is wrapped in the direction of the longitudinal axis “L” extending on to and covering at least a portion of the first surface 34 and at least a portion of the opposed second surface 36 of absorbent material 28 proximal to the insertion end 38. As seen in FIG. 3 the overwrap 40 is “C” wrapped around the insertion end 38 of the absorbent material 28.

As shown in FIG. 3, another overwrap 56 is wrapped in the direction of the longitudinal axis “L” extending on to and covering at least a portion of the first surface 34 and at least a portion of the opposed second surface 36 of absorbent material 28 proximal to the withdrawal end 42 of the absorbent material 28. As seen in FIG. 3 the overwrap 56 is “C” wrapped around the withdrawal end 42 of the absorbent material 28. In the embodiment shown in FIG. 3, the overwrap 40 and over wrap 56 overlap at region 52. The overwrap 56 extends beyond the withdrawal end 42 of the first surface 34 of the absorbent material to form a first skirt portion 46. The overwrap 56 extends beyond the withdrawal end 42 of the second surface 36 of the absorbent material to form a second skirt portion 48. In the embodiment shown, the first skirt portion 46 is joined to the second skirt portion 48 proximal to the withdrawal end 42 of the absorbent material 28 by spot bonds 50.

In some embodiments of making the tampon 20 of the present invention, absorbent material 28 and one overwrap 40 is provided and wrapped around the absorbent material 28 in the direction of the transverse axis “T.” FIG. 4 is a plan view of the tampon 20 of the present invention prior to compression comprising absorbent material 28 and an overwrap 40. The absorbent material 28 has a first surface 34 opposed to a second surface 36 and a first side edge 60 and a second side edge 62. In the embodiment shown, the overwrap 40 is wrapped in the direction of the transverse axis “T” around the first side edge 60 and the second side edge 62 extending on to and covering the first surface 34 and the second surface 36. The seam 32 is shown to the left of the longitudinal axis “L.” In some embodiments, the overwrap 40 overlaps with itself at the seam 32. The overwrap 40 extends beyond the withdrawal end 42 of the first surface 34 of the absorbent material to form a first skirt portion 46. The overwrap 40 extends beyond the withdrawal end 42 of the second surface 36 of the absorbent material to form a second skirt portion 48. In the embodiment shown, the first skirt portion 46 is joined to the second skirt portion 48 proximal to the withdrawal end 42 of the absorbent material 28 by spot bonds 50.

In some embodiments of making the tampon 20 of the present invention, absorbent material 28 and two overwraps 40, 56 are provided and wrapped around the absorbent material 28 in the direction of the transverse axis “T.” FIG. 5 is a plan view of the tampon 20 of the present invention prior to compression comprising absorbent material 28 and two overwraps 40, and 56. The absorbent material 28 has a first surface 34 opposed to a second surface 36 and a first side edge 60 and a second side edge 62. In the embodiment shown, the overwrap 56 is wrapped in the direction of the transverse axis “T” around the first side edge 60 extending on to and covering a portion of the first surface 34 and covers a portion of the opposed second surface 36 of the absorbent material 28. As seen in FIG. 5 the overwrap 56 is “C” wrapped around the first side edge 60 of the absorbent material 28.

As shown in FIG. 5, a second overwrap 40 is wrapped in the direction of the transverse axis “T” around the second side edge 62 extending on to and covering a portion of the first surface 34 and covers a portion of the opposed second surface 36 of the absorbent material 28. As seen in FIG. 5 the overwrap 40 is “C” wrapped around the second side edge 62 of the absorbent material 28. A portion of the overwrap 40 and a portion of the overwrap 56 extends beyond the withdrawal end 42 of the first surface 34 of the absorbent material to form first skirt portion 46. A portion of the overwrap 40 and a portion of the overwrap 56 extends beyond the withdrawal end 42 of the second surface 36 of the absorbent material to form a second skirt portion 48. In the embodiment shown in FIG. 5, the first skirt portion 46 is joined to the second skirt portion 48 proximal to the withdrawal end 42 of the absorbent material 28 by spot bonds 50. The overwrap 40 and the overwrap 56 overlap at region 52.

The absorbent material 28 used in making the tampon 20 of the present invention may be constructed from a wide variety of liquid-absorbing materials commonly used in absorbent articles. Such materials include but are not limited to rayon (such as GALAXY Rayon (a tri-lobed rayon structure) available as 6140 Rayon; or SARILLE L rayon (a round fiber rayon), both available from Acordis Fibers Ltd., of Hollywall, England), cotton, folded tissues, woven materials, nonwoven webs, synthetic and/or natural fibers or sheeting, comminuted wood pulp which is generally referred to as airfelt, or combinations of these materials. Additional absorbent material 28 include materials, such as peat moss, absorbent foams (such as those disclosed in U.S. Pat. No. 3,994,298 issued to DesMarais on Nov. 30, 1976, U.S. Pat. No. 5,795,921 issued to Dyer, et.) capillary channel fibers (such as those disclosed in U.S. Pat. No. 5,356,405 issued to Thompson, et. al), high capacity fibers (such as those disclosed in U.S. Pat. No. 4,044,766 issued Kaczmarzk et al. Aug. 30, 1977), superabsorbent polymers or absorbent gelling materials (such as those disclosed in No. 5,830,543 issued to Miyake, et al) may be incorporated into the tampon 20.

The absorbent material 28 that comprises the compressed absorbent member can be rectangular or any other shape prior to compression and/or shaping. A more detailed description of liquid-absorbing materials shapes and dimensions can be found in co-pending case Ser. No. 10/039,979, filed Oct. 24, 2001, entitled “Improved Protection and Comfort Tampon,” to Agyapong et al., Docket Number 8758.

Overwraps 40 and 56 may be comprised of wovens, nonowovens, or films, which may comprise a blend of natural fibers, synthetic fibers or natural and synthetic fibers. The natural fibers include rayon, cotton, wood pulp, flax, and hemp. The synthetic fibers can include but are not limited to fibers such as polyester, polyolefin, nylon, polypropylene, polyethylene, polyacrylic, vinyl polyacetate, polyacrylate, cellulose acetate or bicomponent fibers. The blend of fibers forming the overwrap can be made by any number of techniques. The blends may be carded on webs. Commonly carded webs that are hydroentangled, thermally bonded, and resin bonded all have application. Spunbond and meltblown processes, combining synthetic fibers extruded/spun onto/into a mat or carded web of natural fibers provide other acceptable techniques. The basis weight of the material may fall into a range from about 10 to about 60 g/m² or typically from about 15 to about 40 g/m².

One embodiment of the overwraps 40 and 56 includes a 100% rayon material, including a 40 g/m² nonwoven comprising 100% rayon available as SX 275-123 produced by Green Bay Nonwovens, Green Bay, Wis. Some embodiments of the overwrap may include blends comprising from about 25% rayon to about 100% rayon. Another embodiments may comprise a 35 g/m 2 hydroentangled blend of 50% rayon, 50% polyester produced by Green Bay Nonwovens, Green Bay, Wis. under the designation SX-367. One embodiment may include a nonwoven comprised of bicomponent fibers that have polypropylene core surrounded by polyethylene manufactured by Vliesstoffwerke Christian Heinrich Sandler GmbH & Co.KG (Schwarzenbach/Saale, Germany) under the tradename SAS B31812000. One embodiment may include a thermally bonded nonwoven of 17 g/m² basis weight manufactured by Vliesstoffwerke Christian Heinrich Sandler GmbH & Co.KG (Schwarzenbach/Saale, Germany) under the tradename Sawabond 4313.

In all embodiments shown, the overwraps 40 and 56 are generally rectangular, but other shapes such as trapezoidal, triangular, hemispherical, chevron, hourglass shaped, “T” and “L” shaped are also acceptable. Optimally, the overwrap 40 and 56 generally may correspond to the shape of the absorbent material 28.

The overwraps 40 and 56 may be joined to the absorbent material 28 by any variety of means. Such joining may extend continuously along the length of attachment or it may be applied in spot bonds in a “dotted” fashion at discrete intervals. Methods of joining include sewing, adhesives, or bonding including thermally bonding, pressure bonding, fusion bonding, ultrasonic bonding or any other suitable means known in the art for joining such materials. Alternatively, the overwrap 40, 56 may be joined to the absorbent material 28 along with the withdrawal means 54 by stitching. Such stitching may use natural or synthetic thread.

The tampon 20 of the present invention may comprise an overwrap 40 or overwraps 40, 56. In some embodiments of the making of the tampon 20 of the present invention, two overwraps 40 and 56 are provided. In some embodiments, each of the two overwraps 40 and 56 may possess or be imparted with different absorbent characteristics. In other embodiments, one overwrap may be imparted with different absorbent characteristics in different regions of the overwrap 40, 56. For example, a tampon with an overwrap or overwraps having both masking and wicking properties is disclosed in U.S. patent application Ser. No. 10/298,403, filed on Nov. 18, 2002. The overwrap 40 or overwraps 40 and 56 may be treated to be hydrophobic or may be treated to render the region or overwrap 40 and 56 hydrophobic if the starting material is hydrophilic. Treatments that render a material hydrophobic include but are not limited to applying to or dipping the material in silicones, fatty acids (such as sucrose ester fatty esters), fluorocarbons, such as SCOTCHGUARD, and waxes. As well, the overwrap 40 or overwraps 40, 56 may be hydrophilic or may treated to render the region or overwrap 40, 56 to be hydrophilic if the starting material is hydrophobic. Treatments that render a material hydrophilic include but are not limited to applying or dipping the material in surfactants, including non-ionic surfactants. The absorbent material 28 used in making the tampon of the present invention may be any suitable size and thickness suitable for compression into a tampon 20 having a vaginally insertable shape. The absorbent material 28 is generally square or rectangular, but other shapes such as trapezoidal, triangular, hemispherical, chevron and hourglass shaped are also acceptable. A typical size for absorbent material 28 prior to compression may be from about 40 mm to about 100 mm in length and from about 40 mm to about 80 mm in width. The typical range for the overall basis weight is from about 150 g/m² to about 1000 g/m².

As shown in FIG. 1-FIG. 5, the tampon 20 of the present invention prior to compression comprises a first skirt portion 46 and a second skirt portion 48 that is formed from the overwrap 40 that extends beyond the withdrawal end 42 of the absorbent material 28. The first skirt portion 46 and the second skirt portion 48 have a width that is proportional to the width of the absorbent material 28 given that the width of the overwrap 40 may be wider or less wide than the measure of the longitudinal axis “L” or transverse axis “T” of the absorbent material 28 it is being wrapped around.

In some embodiments, the first skirt portion 46 and second skirt portion 48 may not be compressed. Both the absorbent material 28 and the first skirt portion 46 and second skirt portion 48 may reside entirely within the vaginal cavity of the wearer during use of the tampon 20. This is achieved by the relative closeness of the first skirt portion 46 and second skirt portion 48 to the withdrawal end 42 of the absorbent material 28 and the relative size compared to the overall size of the tampon 20. In particular embodiments, only the withdrawal means 54 resides externally to the orifice of the vagina.

In some embodiments, the first skirt portion 46 and the second skirt portion 48 may extend from about 2 mm to about 40 mm, or from about 5 mm to about 25 mm beyond the withdrawal end 42 defining the first and second skirt portions. In other embodiments, first skirt portion 46 and second skirt portion 48 may extend from about 10 mm to about 15 mm beyond the withdrawal end 42. As well, the first skirt portion 46 and the second skirt portion 48 may extend from about 15 mm to about 20 mm beyond the withdrawal end.

In all embodiments, the first skirt portion 46 is joined to the second skirt portion 48 proximal to the withdrawal end 42 of the absorbent material 28. Methods of joining include sewing, adhesives, including thermally bonding, pressure bonding, fusion bonding, ultrasonic bonding or any other suitable means known in the art for joining such materials. Such joining may extend continuously along the width of the first skirt portion 46 and the second skirt portion 48 or it may be applied in a plurality of spot bonds in a “dotted” fashion at discrete intervals on the first skirt portion 46 and the second skirt portion 48. The spot bonds 50 may be any geometrical shape including circle, ovals, square, or rectangular. The spot bonds may be any size that would fit inside the first skirt portion 46 and second skirt portion 48. In some embodiments, the spot bonds 50 may be applied or “dotted” randomly or in a pattern, such as straight lines, diagonal lines, triangles, rectangles, circles, diamonds, or any other geometric shape. In some embodiments first skirt portion 46 is joined to the second skirt portion 48 from about 2 mm to about 10 mm from the withdrawal end 42 or about 4 mm to about 8 mm from the withdrawal end of the absorbent material. In some embodiments, the first skirt portion 46 and second skirt portion 48 may be joined from about 1 mm to about 5 mm inward from the first side edge 60 toward the longitudinal axis “L.” As well, the first skirt portion 46 and second skirt portion 48 may be joined from about 1 mm to about 5 mm inward from the second side edge 62 toward the longitudinal axis “L.”

Some embodiments of the tampon 20 of the present invention may comprise a withdrawal means 54. The withdrawal means 54 may be joined to at least the absorbent material 28 and extends beyond at least the withdrawal end 42 of the absorbent material 28. Any of the withdrawal means 54 currently known in the art may be used as a suitable withdrawal mechanism. The withdrawal means 54 may be attached in any suitable manner known in the art including sewing, adhesive attachment, or a combination of known bonding methods.

In some embodiments, the tampon 20 of the present invention may be compressed to a vaginally insertable shape by any method known in the art. Prior to compression, the absorbent material 28 and overwrap 40 may be rolled or folded. Subsequent to compression, the tampon 20 may be heat conditioned in any suitable manner. The tampon 20 of the present invention may be inserted digitally or through the use of any currently available applicator.

All documents cited are, in relevant part, incorporated herein by reference; the citation of any document is not to be construed as an admission that it is prior art with respect to the present invention.

While particular embodiments of the present invention have been illustrated and described, it would be obvious to those skilled in the art that various other changes and modifications can be made without departing from the spirit and scope of the invention.

What is claimed is:

1. A tampon comprising:
an absorbent material having a first surface opposed to the second surface, and an insertion end opposed to a withdrawal end;
an overwrap covering at least a portion of said first surface and at least a portion of said second surface proximal to said withdrawal end of said absorbent material;
said overwrap extends beyond the withdrawal end of said first surface of said absorbent material to define a first skirt portion;
said overwrap extends beyond the withdrawal end of said second surface of said absorbent material to define a second skirt portion; and
said first skirt portion is joined to said second skirt portion proximal to the withdrawal end of the absorbent material.

2. A tampon according to claim 1 wherein said first skirt portion is joined to said second skirt portion by a plurality of spot bonds.

3. A tampon according to claim 1 wherein said first skirt portion and said second skirt portion have a width.

4. A tampon according to claim 3 wherein said first skirt portion is joined to said second skirt portion continuously along the width of said first skirt portion and said second portion.

5. A tampon according to claim 3 wherein said first skirt portion is joined to said second skirt portion continuously along the width of said first skirt portion and said first skirt portion is joined to said second skirt portion by a plurality of spot bonds.

6. A tampon according to claim 1 wherein said first skirt portion and said second skirt portion extend from about 2 mm to about 40 mm from said withdrawal end of said absorbent material.

7. A tampon according to claim 1 wherein said first skirt portion and said second skirt portion extend about 15 mm to about 20 mm from said withdrawal end of said absorbent material.

8. A tampon according to claim 1 wherein said first skirt portion and said second skirt portion are joined from about 4 mm to about 8 mm from said withdrawal end.

9. A tampon according to claim 1 wherein the tampon is compressed.
